(19) United States
(12) Patent Application Publication
(10) Pub. No.: US 2014/0243687 A1
(43) Pub. Date: Aug. 28, 2014

(54) SHAPE SENSING DEVICES FOR REAL-TIME MECHANICAL FUNCTION ASSESSMENT OF AN INTERNAL ORGAN

(71) Applicant: KONINKLIJKE PHILIPS N.V., EINDHOVEN (NL)

(72) Inventors: Bharat Ramachandran, Morganville, NJ (US); Raymond Chan, San Diego, CA (US); Robert Manzke, Bonebuttel (DE)

(73) Assignee: KONINKLIJKE PHILIPS N.V.

(21) Appl. No.: 14/351,940

(22) PCT Filed: Oct. 19, 2012
(86) PCT No.: PCT/IB2012/055742

§ 371 Date: Apr. 15, 2014

Related U.S. Application Data
(60) Provisional application No. 61/549,298, filed on Oct. 20, 2011.

(51) Int. Cl. A61B 5/00; A61B 5/026; A61B 5/11; A61B 5/029; A61B 5/02

(52) U.S. Cl. CPC A61B 5/6847; A61B 5/029; A61B 5/02028; A61B 5/1107; A61B 5/7271; A61B 5/0261
USPC 600/478

(57) ABSTRACT

A system and method for functioning organ assessment include a sensing enabled flexible device (102) having an optical fiber configured to sense induced strain continuously over a length of the flexible device. The flexible device includes a manipulation mechanism (105) configured to permit engagement with an interior wall of an organ over the length. An interpretation module (115) is configured to receive optical signals from the optical fiber between two phases of movement of the organ while the organ is functioning and to interpret the optical signals to quantify parameters associated with the functioning of the organ.

This disclosure relates to shape sensing devices and more particularly to systems and methods for interventional procedures with minimally invasive real-time functioning organ assessment.

Interventional procedures that are performed in a cardiac catheterization laboratory (CathLab) typically include catheters being inserted through blood vessels in the arm, leg or neck and advancing the catheters into the heart. This approach permits access to the heart while the heart is functioning. These procedures can be performed without stopping the heart or requiring highly invasive sternotomy (cutting open the sternum), thoracotomy (cutting through ribcage to access the pleural cavity), etc., and therefore, these procedures minimize potential trauma involved with cardiac interventions. Many interventional procedures such as percutaneous transluminal coronary angioplasty (PTCA), radiofrequency (RF) ablations, drug delivery, cardiac resynchronization therapy (CRT) and myocardial biopsy are performed under X-ray fluoroscopic guidance which entails injecting iodinated contrast through the catheter into the cardiovascular system to temporarily visualize heart function (e.g., blood flow and contraction pattern) and vessel location. Due to its real-time nature, lower costs and absence of tissue damage caused by harmful radiation, research has focused on ultrasound or transesophageal echocardiography (TEE) guided interventional procedures.

Due to advantages over open heart surgery, such as, faster recovery and higher survival rates, the number of minimally-invasive cardiac procedures is increasing. These interventional procedures are typically performed under X-ray fluoroscopy guidance that gives the interventionalist limited information about the anatomy and function of the heart. While potentially being harmful to the physician and patient, interventional X-ray imaging only provides limited information about the anatomy and function due to the limited soft-tissue contrast available. For this reason, research has focused on adding other imaging modalities to the clinical workflow in the interventional lab. The use of intra-procedural ultrasound, for example, potentially allows for mechanical function assessment such as wall motion and cardiac output. Drawbacks of using ultrasound include restricted field of view, low signal to noise ratio (SNR), and subjective techniques, which are very prone to inter-sonographer variations in scanning ability and image interpretation. Electromagnetic tracking techniques to provide real-time position information of the catheter suffer from difficulties in obtaining motion and function estimates of the myocardium, since tracking is limited to a very sparse set of discrete measurement locations (typically no more than 5 sensor locations).

In accordance with the present principles, a system and method for functioning organ assessment include a sensing enabled flexible device having an optical fiber configured to sense induced strain continuously over a length of the flexible device. The flexible device includes a manipulation mechanism configured to permit engagement with an interior wall of an organ over the length. An interpretation module is configured to receive optical signals from the optical fiber between two phases of movement of the organ while the organ is functioning and to interpret the optical signals to quantify parameters associated with the functioning of the organ.

A workstation for functional heart assessment includes a processor, memory coupled to the processor and a sensing enabled flexible device having at least one optical fiber configured to sense induced strain continuously over a length of the flexible device. A manipulation mechanism is integrated into the flexible device and configured to permit engagement of the flexible device with a wall and/or vessel of a heart over the length. An interpretation module is stored in the memory and configured to receive feedback signals from the at least one optical fiber between at least two phases of movement of the heart while the heart is functioning. The interpretation module generates data to quantify parameters associated with the functioning of the heart based on the induced strain. A display is configured to generate images to assist in performing a procedure on the functioning heart.

A method includes inserting a sensing enabled flexible device having at least one optical fiber configured to sense induced strain continuously over a length of the flexible device into a chamber or vessel of a functioning organ; manipulating the flexible device to engage with boundaries of the organ over the length; receiving feedback signals from the at least one optical fiber for at least two phases of movement of the organ while the organ is functioning; and interpreting the feedback signals to quantify parameters associated with the functioning of the organ.

These and other objects, features and advantages of the present disclosure will become apparent from the following detailed description of illustrative embodiments thereof, which is to be read in connection with the accompanying drawings.

This disclosure will present in detail the following description of preferred embodiments with reference to the following figures wherein:

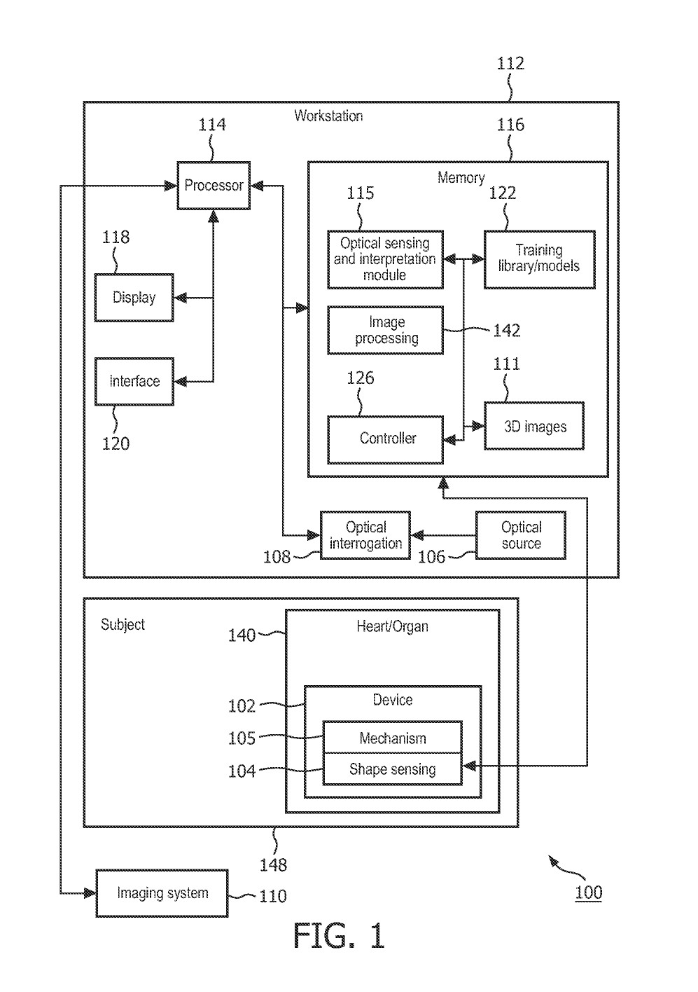

FIG. 1 is a block/flow diagram showing a system/method for assessing functional organs in accordance with the present principles;

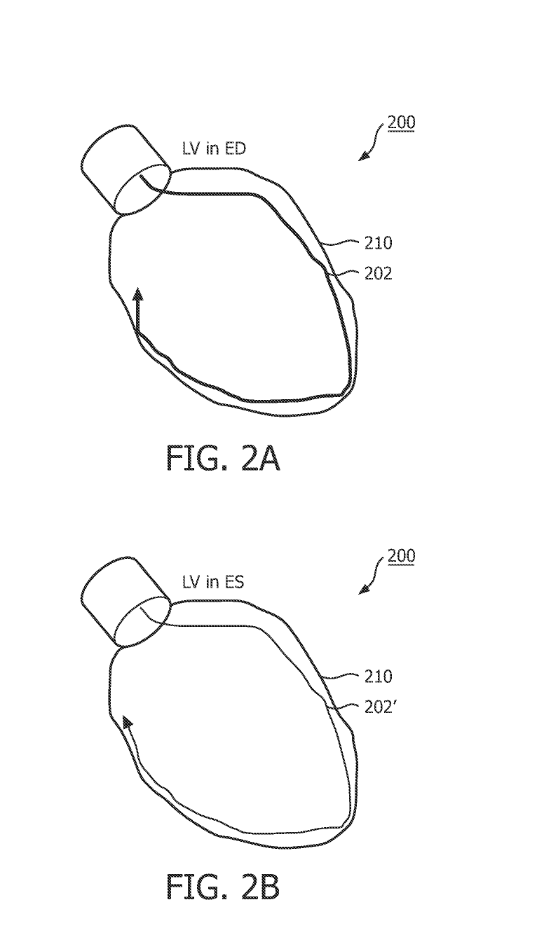

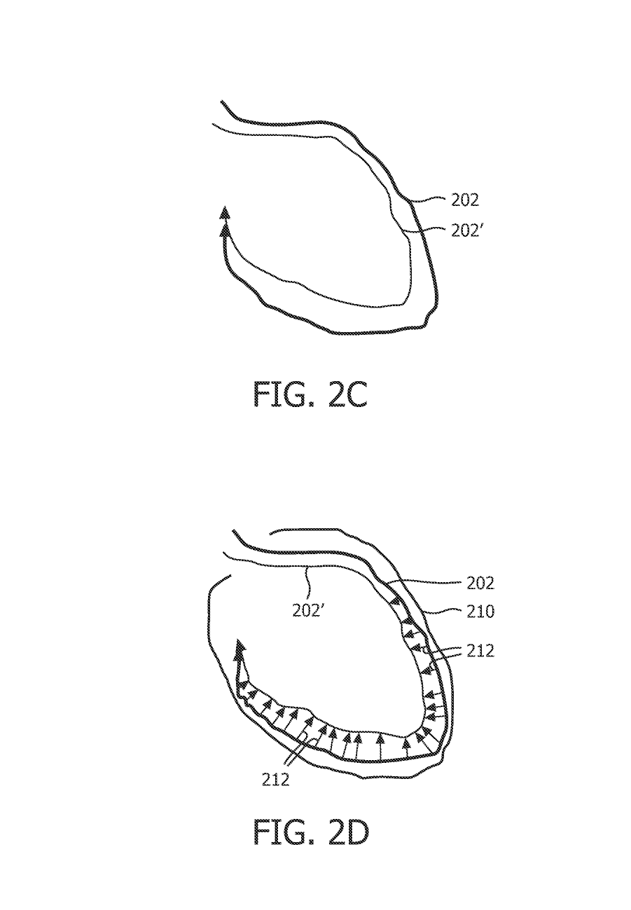

FIG. 2A is a diagram showing shape sensing of a left ventricle of a heart in an end diastole (ED) position in accordance with an exemplary procedure;

FIG. 2B is a diagram showing shape sensing of the left ventricle of the heart in an end systole (ES) position in accordance with an exemplary procedure;

FIG. 2C shows theorized positions of a catheter or catheters of FIGS. 2A and 2B overlaid on each other showing motion of a myocardium during a cardiac cycle in accordance with one embodiment;

FIG. 2D shows arrows quantifying a displacement between end diastole and end systole for the left ventricle of FIG. 2C; and

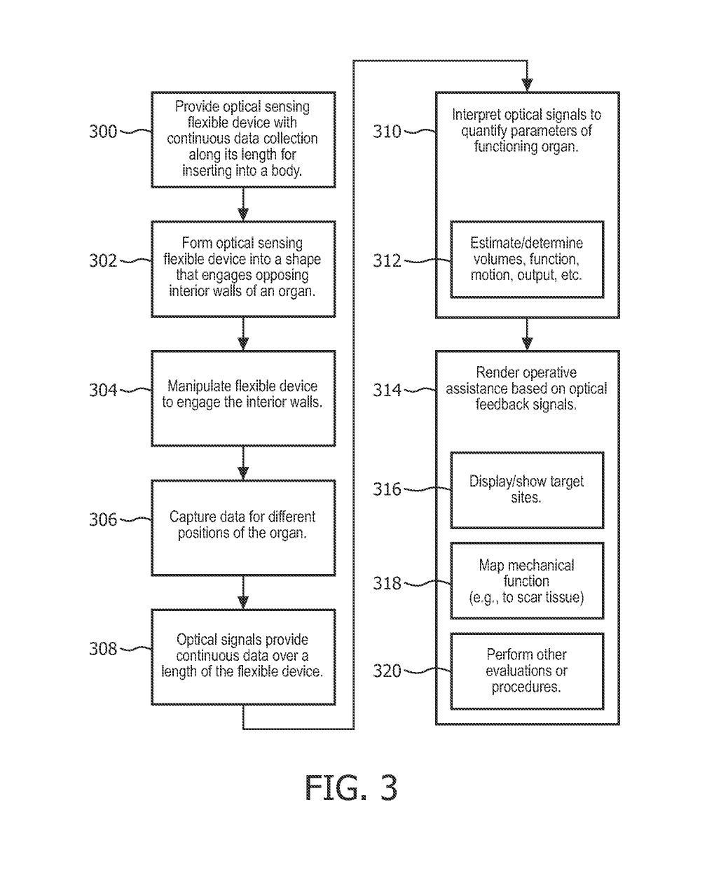

FIG. 3 is a flow diagram showing steps for assessing a functioning organ in accordance with an illustrative embodiment of the present invention.

In accordance with the present principles, continuous spatial and temporal measurement of a boundary permits real-time mechanical function assessment of the heart as well as verification of success of a cardiac interventional procedure such as cardiac resynchronization therapy (CRT). In one embodiment, an optical shape sensing enabled flexible device (such as catheters, guidewires, leads, etc.) is included to perform continuous real-time motion and function assessment of the heart or other organ. In accordance with the present principles, the embodiments can provide information such as mechanical dyssynchrony or other phenomena through direct interrogation of motion, myocardial viability and cardiac output through indirect estimates derived from motion characteristics measured during an interventional procedure. Instead of discrete optical sensors, the present principles provide for spatially and temporally continuous sensing of distributed parameters along a known three-dimensional (3D) path. This information is needed for optimizing clinical outcomes. For example, direct mechanical feedback about cardiac pacing protocols would be possible with this continuous information during a pacing optimization intervention, helping with proper lead placement in the heart.

In particularly useful embodiments, systems and methods provided herein permit real-time mechanical function assessment of the heart during a cardiac catheterization procedure by adding optical shape sensing along the length of the catheter or other device. Data is acquired in real-time while positioning a distal portion of the catheter along the inner walls of the myocardium throughout a cardiac cycle. This permits rapid interrogation of the motion (contraction and relaxation) along the walls of the chambers in three dimensions, allowing for on-line “live” computation of cardiac volumes, ejection fraction and cardiac output as well as detecting the patterns of motion. This real-time function information of the heart can be used to verify the extent of success of pacemaker lead implantations for CRT or other procedures.

In addition to interventional cardiac procedures and intra-operative functional assessment of the heart, the present principles may be employed to validate functional imaging of organs, make curved or linear measurements within a functioning organ, provide suitability studies for placements of treatment devices, (e.g., pacemaker leads, etc.) among other applications.

It should be understood that the present invention will be described in terms of medical instruments; however, the teachings of the present invention are much broader and are applicable to any instruments employed in tracking or analyzing complex biological or mechanical systems. In particular, the present principles are applicable to internal tracking procedures of biological systems, procedures in all areas of the body such as the lungs, gastro-intestinal tract, excretory organs, blood vessels, etc. The elements depicted in the FIGS. may be implemented in various combinations of hardware and software and provide functions which may be combined in a single element or multiple elements.

The functions of the various elements shown in the FIGS. can be provided through the use of dedicated hardware as well as hardware capable of executing software in association with appropriate software. When provided by a processor, the functions can be provided by a single dedicated processor, by a single shared processor, or by a plurality of individual processors, some of which can be shared. Moreover, explicit use of the term “processor” or “controller” should not be construed to refer exclusively to hardware capable of executing software, and can implicitly include, without limitation, digital signal processor (“DSP”) hardware, read-only memory (“ROM”) for storing software, random access memory (“RAM”), non-volatile storage, etc.

Moreover, all statements herein reciting principles, aspects, and embodiments of the invention, as well as specific examples thereof, are intended to encompass both structural and functional equivalents thereof. Additionally, it is intended that such equivalents include both currently known equivalents as well as equivalents developed in the future (i.e., any elements developed that perform the same function, regardless of structure). Thus, for example, it will be appreciated by those skilled in the art that the block diagrams presented herein represent conceptual views of illustrative system components and/or circuitry embodying the principles of the invention. Similarly, it will be appreciated that any flow charts, flow diagrams and the like represent various processes which may be substantially represented in computer readable storage media and so executed by a computer or processor, whether or not such computer or processor is explicitly shown.

Furthermore, embodiments of the present invention can take the form of a computer program product accessible from a computer-usable or computer-readable storage medium providing program code for use by or in connection with a computer or any instruction execution system. For the purposes of this description, a computer-usable or computer readable storage medium can be any apparatus that may include, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. Examples of a computer-readable medium include a semiconductor or solid state memory, magnetic tape, a removable computer diskette, a random access memory (RAM), a read-only memory (ROM), a rigid magnetic disk and an optical disk. Current examples of optical disks include compact disk—read only memory (CD-ROM), compact disk—read/write (CD-R/W) and DVD.

Referring now to the drawings in which like numerals represent the same or similar elements and initially to FIG. 1, a system 100 for performing a medical procedure is illustratively depicted. System 100 may include a workstation or console 112 from which a procedure is supervised and managed. Procedures may include any procedure including but not limited to cardiovascular procedures, vascular procedures, bronchial procedures, etc. Workstation 112 preferably includes one or more processors 114 and memory 116 for storing programs and applications. It should be understood that the function and components of system 100 may be integrated into one or more workstations or systems.

Memory 116 may store an optical sensing and interpretation module 115 configured to interpret optical feedback signals from a shape sensing device 104. Optical sensing module 115 is configured to use the optical feedback signals (and any other feedback, e.g., electromagnetic (EM)) to reconstruct deformations, deflections and other changes associated with a medical device 102 and/or its surrounding region. The medical device 102 preferably includes an elongated device and may include, e.g., a catheter, a guide wire, a lead wire, an endoscope, a probe, a robot, an electrode, a filter device, a balloon device, or other medical component, etc. In a particularly useful embodiment, device 102 includes a catheter, a guide wire or a lead wire configured for interventional heart procedures.

Workstation 112 may include a display 118 for viewing internal images of a subject if an imaging system 110 is employed. The imaging system 110 may include, e.g., a magnetic resonance imaging (MRI) system, a fluoroscopy system, a computed tomography (CT) system, etc. Display 118 may also permit a user to interact with the workstation 112 and its components and functions. This is further facilitated by an interface 120 which may include a keyboard, mouse, a joystick or any other peripheral or control to permit user interaction with the workstation 112.

A controller 126 may be included in a software module or may include manual controls for controlling and/or maneuvering the device 102. Controller 126 may control a manipulation mechanism 105 integrated into the flexible device 102 and configured to permit engagement of the flexible device 102 with a wall and/or vessel of an organ. Manipulation mechanism 105 may include wires, guides, pressures, etc. needed to steer or guide the device 102. These mechanisms and controller 126 are also employed in placement of the device 102 on boundaries of chambers or the like as will be described herein.

Workstation 112 includes an optical source 106 to provide optical fibers with light. An optical interrogation unit 108 is employed to detect light returning from all fibers. This permits the determination of strains or other parameters, which will be used to interpret the shape, orientation, etc. of the interventional device 102. The light signals will be employed as feedback to make adjustments to access errors and to calibrate the device 102 or system 100.

Shape sensing device 104 includes one or more fibers which are configured to exploit their geometry for detection and correction/calibration of a shape of the device 102. Optical interrogation unit/module 108 works with optical sensing module 115 (e.g., shape determination program) to permit tracking of instrument or device 102. Shape sensing with fiber optics may be based on fiber optic Bragg grating sensors. A fiber optic Bragg grating (FBG) is a short segment of optical fiber that reflects particular wavelengths of light and transmits all others. This is achieved by adding a periodic variation of the refractive index in the fiber core, which generates a wavelength-specific dielectric mirror. A fiber Bragg grating can therefore be used as an inline optical filter to block certain wavelengths, or as a wavelength-specific reflector. A principle behind the operation of a fiber Bragg grating is Fresnel reflection at each of the interfaces where the refractive index is changing. For some wavelengths, the reflected light of the various periods is in phase with one another so that constructive interference exists for reflection and consequently, destructive interference for transmission. The Bragg wavelength is sensitive to strain as well as to temperature. The Bragg gratings can be used as sensing elements in fiber optical sensors.

One of the main advantages of this technique is that various sensor elements can be distributed over the length of a fiber. Incorporating three or more cores with various sensors (gauges) along the length of a fiber that is embedded in a structure allows for the three dimensional form of such a structure to be precisely determined. Along the length of the fiber, at various positions, a multitude of FBG sensors are located (e.g., three or more fiber sensing cores). From the strain measurement of each FBG the curvature of the structure can be inferred at that position. From the multitude of measured positions, the total three dimensional form is determined.

As an alternative to fiber optic Bragg gratings, the inherent backscatter in an optical fiber, can be exploited. One such approach is to use Rayleigh scatter in standard single-mode communications fiber. Rayleigh scatter and/or Brillouin scatter occurs as a result of random fluctuations of the index of refraction in the fiber core. These random fluctuations can be modeled as a Bragg grating with a random variation of amplitude and phase along the grating length. By using this effect in three or more cores running within a single length of multicore fiber, the 3D shape and dynamics of the surface of interest would be trackable. The use of scattering permits continuous monitoring over an entire length of an optical fiber. In accordance with the present principles, FBGs may be employed but Raleigh scattering or Brillouin scattering is preferable for cardiac procedures as will be described herein.

In a particularly useful embodiment, device 102 is employed to discover or observe a target. The target may include a functioning organ, such as the heart, lungs, etc. During a procedure, shape sensing data from shape sensing device 104 is collected and registered with pre-operative imaging data or previously collected shape sensing data to understand real-time functioning of the target. The shape sensing data may include motion data from a heartbeat and/or breathing, and an analysis may be performed to account for the same.

In one embodiment, the device 102 having the shape sensing device 104 with Rayleigh, FBG and/or Brillouin scattering capabilities permits rapid detection of the shape changes of chambers within the heart along a known 3D path and subsequent estimation of cardiac volumes and mechanical functions. Module 115 employs dynamic shape sensing data to compute cardiac parameters based on statistical models/training libraries 122 and represents the parameters on the display 118 during the intervention. The module 115 interprets the shape sensing data to suggest target sites for pacer implantation based on shape sensing mechanical function data and further pre- or intra-operative data. The module 115 can also map mechanical function of the heart to scar-locations due to pathologic tissue deformation patterns.

An imaging system 110 may be provided for collecting pre-operative imaging data or real-time intra-operative imaging data of a subject 148. The pre-operative imaging may be performed at another facility, location, etc. in advance of any procedure. 3D images 111 may be stored in memory 116 and employed with an output of module 115 to visualize placement of the shape sensing device 104 and further to indicate, as an overlay, areas of treatment, wire placement locations for CRT, areas free of scar tissue or other parameters or computed features consistent with the medical procedure and organ of interest.

The system 100 can thus, provide a clinician valuable information (e.g., the location of the myocardial boundary) about a myocardial surface, without the need for X-ray imaging or contrast injection, which would be needed in other techniques, such as discrete measurement techniques where discrete (point) readings are made as a function of length along a tether. The present system 100 employs optical shape sensing fiber 104 integrated into a flexible instrument 102 (such as a catheter, guidewire, pressure wire, or electrode lead wire) to provide continuous spatio-temporal information in three dimensions.

During the procedure, the shape tracked flexible instrument 102 is positioned next to the walls of a heart 140 (or other organ) using a standard maneuver such as one employed for electrophysiology interventions in which the catheter/wire 102 is looped so that it encircles an epi- or endocardial boundary. Repeating this procedure with simultaneous probing by several shape tracked flexible instruments (102), or with sequential probing with a single tracked instrument to interrogate different cut-planes within the cardiac chamber, permits demarcation of the boundary between the myocardium and a chamber, which cannot presently be determined without contrast injection during conventional X-ray fluoroscopic/cineangiographic guidance.

In addition, the shape sensing enabled flexible instrument 102 could feed contour/boundary data as well as motion measurements into an image processing module 142 for registration, segmentation, reconstruction, or quantitation to automate algorithms that would otherwise require clinical input in the form of seeding contours that are manually defined based on visual interpretation of imaging data. In this case, the flexible instrument 102 provides input seed measurements and acts, in a sense, as if it were a human-computing interface device (e.g., a mouse).

Referring to FIGS. 2A-2D, shape sensing of a heart chamber is illustratively shown in accordance with an exemplary procedure. FIG. 2A shows a diagram of a left ventricle (LV) 200 in end diastole (ED) position. A shape sensing catheter 202 is inserted and positioned adjacent to an inner myocardial surface 210. FIG. 2B shows the LV 200 in end systole (ES) position and the catheter 202′ adjacent to the inner boundary 210. FIG. 2C shows theorized positions of the catheter or catheters 202 (202′), with one overlaid on the other showing the motion of the myocardium during a cardiac cycle. The inner catheter position is designated as 202′ to indicate that the image is from FIG. 2B. FIG. 2D shows arrows 212 quantifying a displacement between end diastole and end systole for the LV 200. This technique may be employed for evaluating motion that different regions of the LV 200 are undergoing and any relation between the motions.

Shape sensing-based real-time tracking of the motion of the flexible instrument (e.g., catheter 102, 202, 202′) can be used to derive the motion characteristics of the myocardial segments in contact with the flexible instrument. A three-dimensional (3D) motion model can be built up from several sequential cardiac cycles between which the instrument 202 is manipulated to interrogate the motion behavior of the heart in a variety of different cutplanes.

The scope of the system can be extended further by detecting the regions of increased or reduced movement in the myocardium. This permits the system to detect or verify ischemic regions (scar tissue) intra-operatively, and, if necessary, update or correct a site of pacer lead implantation, myocardial biopsy, alcohol ablation or other targeted therapy.

The shape sensing enabled catheter 202 can be pre-shaped in a ‘U’ or ‘V’ geometry or may employ a balloon or other biasing device to make contact with organ surfaces. The shape sensing enabled catheter 202 is placed adjacent to the walls of the heart or other organ while the heart is functioning. Internal mechanisms (e.g., steering or rigidity control) in the catheter 202 permit for control of tight contact with myocardial tissues. Determining the relation or correlation (or lack thereof), between the motion patterns of opposing heart walls permits the system 100 to give real-time dyssynchrony information which may be employed to select, validate or discard a potential site for lead placement in CRT, for example.

In the example shown in FIGS. 2A-2D, estimation of end-systolic and end-diastolic volumes is depicted. However, other parameters may be determined in the same way, for example, ejection fraction, cardiac volume and output, etc., which all provide real-time function information of the heart. When positioned adjacent to the inner wall of the left ventricle (LV), the shape sensing fibers give the boundary and shape of the chamber from a defined cut-plane or viewpoint. Using measurements from standard cut-planes or perspectives, the system can derive lengths along the major and minor axes and in turn estimate the cardiac functions or parameters of interest. Using shape sensing technology and more particularly Rayleigh or Brillouin backscatter (although FBG technology may also be employed), data over a continuous segment of the catheter 202 can be achieved. This means that there are no dead spots or discrete data collection points. Data is collected over the entire length on the catheter 202 over a continuous time scale. This provides a more complete data set, which results in better medical assessments, better medical decision making and immediate feedback.

The data collected from a functioning heart may be interpreted using models or other mechanisms, such as formulas, software analysis tools, etc. by employing, e.g., module 115 (FIG. 1). As an example, to describe how such measurements would be used to derive a volume, consider the following. A volume can be computed using the known Simpson's rule where the area is computed for each circular slice, and this is integrated for the whole major axis length (L where h=L/3) to find the volume. Alternatively, the known modified Simpson's rule may be employed where a circular area is calculated at three different levels namely mitral valve (A₁), papillary muscle (A₂), apex (A₃), and volume (V) is computed as in the following equation.

$$V = \{ ( ( {A}_{1} + {A}_{2} ) \times h ) \} + \{ ( {A}_{3} ) \times \frac{h}{2} \} + \{ \frac{π {h}^{3}}{6} \} .$$

Other models, formulae and analysis programs may also be employed.

In accordance with the present principles, effectiveness of a procedure can be evaluated immediately by the clinician. For example, in the case of alcohol ablation, which has an impact within minutes, the clinician can decide whether the procedure has had the expected impact and extent, and, if not, allow for correction during the same procedure, rather than waiting for a post-procedural scan followed by repeat intervention at a future date. Similarly, in case of dyssynchrony, the interventionalist can conclude that the position of pacemaker leads has not had a desired effect and thus, reposition the leads at some other part of the myocardium. As a result, the interventionalist would know if the procedure has failed, and would be able to make corrections without leaving the catheterization laboratory setting.

In other embodiments, other organs may be evaluated or studied in accordance with the present principles. For example, a shape sensing enabled flexible instrument 102, 202 could be placed within specific locations of a peripheral vascular system. Thus, constrained to the local anatomy, the flexible instrument 102, 202 would also follow vascular deformations. Cardiovascular parameters such as mechanical function, arterial pulse wave velocity, or vascular distension, etc. can similarly be derived and used for intra-operative guidance and decision making.

Referring to FIG. 3, a block/flow diagram shows a system/method for assessing a functioning organ, in particular the heart in accordance with the present principles. In block 300, a sensing enabled flexible device having at least one optical fiber configured to sense induced strain continuously over a length of the flexible device is inserted into a chamber of a functioning organ. The sensing enabled flexible device may include a catheter, a guidewire, a pressure wire or electrode lead wire and preferably provide continuous spatio-temporal information in three dimensions. The chamber may include a chamber of the heart, a vascular structure, a portion of the lungs, etc. Where the organ includes the heart, the interior wall may include an endocardial boundary or an epicardial boundary.

In block 302, the sensing enabled flexible device is formed into a U or V shaped configuration or a balloon shaped configuration in the chamber preferably along a cut-plane to measure displacement on opposing walls of the organ.

In block 304, the flexible device is manipulated to engage with an interior wall of the organ over the length. The manipulation may employ steering or rigidity controls known in the art for use with catheters and the like. In block 306, optical signals from the at least one optical fiber are received for at least two phases of movement of the organ while the organ is functioning. The at least two phases of movement may include diastolic and systolic positions of the heart. In block 308, the optical signals include continuous backscattered light over the active length of the flexible device to provide continuous data over the length.

In block 310, the optical signals are interpreted to quantify parameters associated with the functioning of the organ. In block 312, the interpretation of optical signals may be employed for one or more of estimating of cardiac volumes, determining mechanical function, determining motion characteristics, determining ejection fraction, determining cardiac output, etc.

In block 314, operative assistance is provided based on optical feedback from the flexible device. This may include comparing data to models, previously collected data, statistics, computing parameters based on formulas or software analysis packages, etc. In block 316, target sites for a pacer implantation may be suggested based on shape sensing mechanical function data. In block 318, the mechanical function data is mapped to scar locations using pathologic tissue deformation patterns. In block 320, other evaluations, assessments etc. or other procedures may be carried out.

In interpreting the appended claims, it should be understood that:
a) the word “comprising” does not exclude the presence of other elements or acts than those listed in a given claim;
b) the word “a” or “an” preceding an element does not exclude the presence of a plurality of such elements;
c) any reference signs in the claims do not limit their scope;
d) several “means” may be represented by the same item or hardware or software implemented structure or function; and
e) no specific sequence of acts is intended to be required unless specifically indicated.

Having described preferred embodiments for shape sensing devices for real-time mechanical function assessment of an internal organ (which are intended to be illustrative and not limiting), it is noted that modifications and variations can be made by persons skilled in the art in light of the above teachings. It is therefore to be understood that changes may be made in the particular embodiments of the disclosure disclosed which are within the scope of the embodiments disclosed herein as outlined by the appended claims.

Having thus described the details and particularity required by the patent laws, what is claimed and desired protected by Letters Patent is set forth in the appended claims:

1. A system, comprising:
a sensing enabled flexible device (102) having at least one optical fiber configured to sense induced strain over a continuous length of the flexible device, the flexible device including a manipulation mechanism (105) configured to permit engagement with a wall of an organ over the length; and
an interpretation module (115) configured to receive optical signals from the at least one optical fiber between at least two phases of movement of the organ while the organ is functioning and interpret the optical signals to quantify parameters associated with the functioning of the organ.

2. The system as recited in claim 1, wherein the organ includes the heart and the wall includes an endocardial or epicardial boundary.

3. The system as recited in claim 2, wherein the at least two phases of movement include diastolic and systolic positions of the heart.

4. The system as recited in claim 1, wherein sensing enabled flexible device (102) includes a flexible elongated instrument and provides continuous spatio-temporal information in three dimensions.

5. The system as recited in claim 1, wherein the parameters include one or more of estimation of cardiac volumes, mechanical function, motion characteristics, ejection fraction, and/or cardiac output.

6. The system as recited in claim 1, further comprising a display (118) wherein the interpretation module (115) is configured to suggest target sites for a pacer implantation based on shape sensing mechanical function data.

7. The system as recited in claim 6, wherein the interpretation module (115) maps the mechanical function data to scar locations due to pathologic tissue deformation patterns.

8. The system as recited in claim 1, wherein the sensing enabled flexible device (102) includes at least one of a balloon, a U shaped configuration or V shaped configuration in a cut-plane to measure displacement on opposing walls of the organ.

9. The system as recited in claim 1, wherein the induced strain sensed over the continuous length includes backscatter measured over the length.

10. A workstation for functional heart assessment, comprising:
a processor (114);
memory (116) coupled to the processor;
a sensing enabled flexible device (102) having at least one optical fiber configured to sense induced strain over a continuous length of the flexible device;
a manipulation mechanism (105) integrated into the flexible device and configured to permit engagement of the flexible device with a wall and/or vessel of a heart over the length;
an interpretation module (115) stored in the memory and configured to receive feedback signals from the at least one optical fiber between at least two phases of movement of the heart while the heart is functioning, the interpretation module generating data to quantify parameters associated with the functioning of the heart based on the induced strain; and
a display (118) configured to generate images to assist in performing a procedure on the functioning heart.

11. (canceled)

12. (canceled)

13. (canceled)

14. (canceled)

15. (canceled)

16. (canceled)

17. (canceled)

18. (canceled)

19. A method, comprising:
inserting (300) a sensing enabled flexible device having at least one optical fiber configured to sense induced strain over a continuous length of the flexible device into a chamber or vessel of a functioning organ;
manipulating (304) the flexible device to engage with boundaries of the organ over the length;
receiving (306) feedback signals from the at least one optical fiber for at least two phases of movement of the organ while the organ is functioning; and
interpreting (310) the feedback signals to quantify parameters associated with the functioning of the organ.

20. (canceled)

21. (canceled)

22. The method as recited in claim 19 wherein the sensing enabled flexible device includes a flexible elongated instrument and provides continuous spatio-temporal information in three dimensions.

23. The method as recited in claim 19, wherein interpreting (310) the optical signals to quantify parameters includes one or more of estimating (312) of cardiac volumes, determining mechanical function, determining motion characteristics, determining ejection fraction, and/or determining cardiac output.

24. (canceled)

25. (canceled)

26. The method as recited in claim 19, further comprising forming (302) the sensing enabled flexible device into a U, V or balloon shaped configuration in a cut-plane to measure displacement on opposing walls of the target.

27. The method as recited in claim 19, wherein receiving optical signals includes receiving (308) continuous backscattered light over the length to provide continuous data over the length.

28. (canceled)
